(19) United States
(12) Patent Application Publication
(10) Pub. No.: US 2010/0125225 A1
(43) Pub. Date: May 20, 2010

(54) System for selective ultrasonic ablation

(76) Inventors: Daniel Gelbart, Vancouver (CA); Samuel Victor Lichtenstein, Vancouver (CA)

Correspondence Address: DANIEL GELBART, 4706 DRUMMOND DR, VANCOUVER, BC, V6T-1B4, CA

(21) Appl. No.: 12/292,424

(22) Filed: Nov. 19, 2008

(51) Int. Cl. A61N 7/02

(52) U.S. Cl. 601/3

(57) ABSTRACT

The invention can selectively heat a diseased area, such as a tumor, in the body while minimizing heating of healthy surrounding tissue. This is done by exposing the undesired tissue to a scanning focused ultrasound beam arriving from different angular directions at different times, all directions passing through the undesired tissue. The system can scan the target area with low power ultrasound, and then activate the higher power over the selected target areas.

### FIELD OF THE INVENTION

The invention relates to the medical field and in particular to the treatment of cancer and prostate problems.

### BACKGROUND OF THE INVENTION

In many diseases it is desired to destroy or affect a non-desired tissue without harming the adjacent normal tissue. A non surgical approach has many advantages, such as shorter recovery time and shorter treatment time. Common non surgical approaches are:
Radiation therapy using X-ray or radioactive materials.
Ultrasound, RF or microwave ablation using a probe applied from the outside or inside of the body, with or without cooling.
Use of drugs that selectively attach themselves or act on the malignant tissue.

Examples of the need for such non-surgical procedure are the destruction of tumors, shrinking of an enlarged prostate and collapsing of diseased parts of a lung affected by emphysema. The use of high intensity focused ultrasound (HIFU) is well known. Ultrasound destroys the undesirable tissue be creating heat and also mechanical damage to the cells. In this disclosure the terms “heating” and “ablation” are used interchangeably and the word “heating” should be broadly interpreted as any mechanism of coupling energy into a tissue, whether the main reaction is heating or not. One advantage of ultrasound is that it can be finely focused into a small spot, as the wavelength of the commonly used ultrasound in such applications is on the order of one mm. A common problem is the need to focus a high energy beam on the non-desired, or diseased, tissue without causing damage to healthy tissue. Since healthy tissue should not be subjected to temperatures much above 40° C. and the undesired tissue should ideally be heated to over 60° C., the increase of temperature in the undesired tissue has to be many times higher than the surrounding tissue (23 degrees vs. 3 degrees in this example when body temperature is 37° C.). Sometimes it is required to heat up the undesired tissue to as high as 90° C. In such a case the temperature increase of the undesired tissue, 53°, is almost 20 times higher as the healthy tissue. Prior art attempts to overcome the problem were local cooling and the superposition of two or more sources of ultrasound. Local cooling can only protect a thin layer of tissue, because of the poor heat conductivity of tissue. The superposition of two or more sources is disclosed in US patent 2005/0038339. When two sources are focused on the same spot the heating of the healthy tissue is reduced by a factor of 2, as the malignant or otherwise undesired tissue is exposed to both beams while the adjacent tissue is exposed only to one beam, assuming beams overlap only over the target area. In the previous example it was shown that the undesired tissue will require from seven to twenty times the temperature increase of the desired tissue (23-53 degrees vs. 3 degrees). Even after allowing for the fact that the beam is more concentrated over the target area, a large number of beams will be required to achieve the desired ratio. This is particularly true for treating enlarged prostates and prostate cancer, where the transition from heated to non-heated area has to be very sharp. Such an array of many transducers is bulky and expensive. The invention allows achieving the equivalent of a very large number of beams using the simplicity and low cost of a single beam. Also, because of the fixed mechanical constraints of US patent 2005/0038339, a system configured for breast cancer (as in the patent) will not be suitable for prostate cancer. A system according to the present invention can be used to treat any part of the body simply by setting up a different scan pattern.

### SUMMARY OF THE INVENTION

The invention can selectively heat a diseased area, such as a tumor, in the body while minimizing heating of healthy surrounding tissue. This is done by exposing the undesired tissue to a scanning focused ultrasound beam arriving from different angular directions at different times, all directions passing through the undesired tissue. The system can scan the target area with low power ultrasound, and then activate the higher power over the selected target areas.

### BRIEF DESCRIPTION OF THE DRAWINGS

FIG. 1 is a cross section of a prostate being exposed to directional high intensity ultrasound.

FIG. 2 is a schematic view of an ultrasound treatment system using a robotic manipulator.

FIG. 3 is a view of the display system showing the results of the scanning and the outline of the area to be ablated.

### DETAILED DISCLOSURE

One aspect of the invention is maximizing the heating of the diseased (or otherwise undesirable) tissue while minimizing heating of the surrounding tissue. This can be achieved by moving around the energy source in order for the heat creating beam to arrive from different directions at different times, all directions having a common point of intersection that is located within the diseased tissue. If all these directions pass through the diseased tissue, the diseased tissue will be heated continuously while the surrounding tissue will be heated intermittently. A similar method is employed today in radiation therapy for cancer; however using heat energy has a significant advantage: the effect or radiation, such as X-ray or radioactivity, is cumulative while the effect of heating is non-cumulative. Heating a tissue by 30 degrees will permanently change it, while heating it 10 times by 3 degrees will have no effect. In the case of radiation the effect will be cumulative. The non-cumulative nature of heating allows the re-use of the same direction after the heat dissipated, a process taking from seconds to minutes.

Another aspect of the invention is the need to match the area and depth of the heated area inside the body, which corresponds to the focused spot size and depth of focus, to the treated organ. In case of small organs such as a prostate the heated area can be a few cubic centimeters and it has to be defined with a accuracy of a few millimeters, with the temperature falling off from, by the way of example, from 60° C.-90° C. degrees to 40° C. over a few millimeters. This is difficult to achieve with RF, microwave or other energy forms but relatively easy to achieve with high intensity focused ultrasound. The focused spot size is related to wavelength, transducer size and tissue properties. It is easiest to express the relationship as a function of the f/# of the transducer, f/# being the ratio of the focal length to the diameter of the transducer. The size of the focused spot is approximately 1.2f/#×wavelength and the depth of the focused zone is approximately 3(f/#)²×wavelength. The common frequencies used for high intensity ultrasound are from 0.5 MHz to 5 MHz, with corresponding wavelengths of about 1.3 mm-0.13 mm. Using these numbers and an f/1 transducer a focused spot of 0.2 mm-2 mm can be achieved. A low f/# is desired not only for achieving a small focused spot but also to ensure that the power density becomes large only in the vicinity of the focused spot. The “depth of focus” in this example, defining the depth of the heated tissue, will be about 0.6 to 6 mm, depending on the frequency used. By selecting the f/#, a large degree of control is possible. More advanced beam shaping techniques can be used to improve these figures. For example, apodizing can greatly increase the focal depth. Clearly the focused spot can be created from a planar transducer by using an acoustic focusing lens of a phased array transducer. The alternative is to use a spherical or parabolic transducer. Such a transducer usually comprises a plurality of smaller transducers operating in parallel.

A third aspect of the invention is the ability to use the high intensity transducer at a lower intensity in order to scan the treatment area and create a 3D map of the area. This can be done by using the same transducer or by a second, co-located transducer. Such an arrangement eliminates any position offset errors between the diagnostic scanning system and the treatment system. Any position offset error between such systems will cause a shift between the desired treatment volume and the actual one.

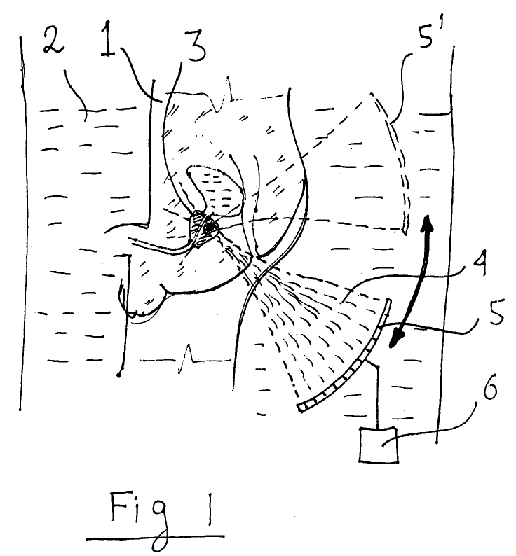

The method of taking advantage of the non-cumulative heating effect is shown in FIG. 1. A patient 1 is placed in a water tank 2 with at least part of the body submerged. A parabolic transducer 5 converts electrical energy fro source 6 into a high intensity focused ultrasonic beam 4, coming to a focus on the area to be heated or ablated such as prostate 3. In order to heat the prostate the acoustic beam 4 has to pass healthy tissue that will heat up as well. By continuously moving transducer 4, as shown by new position 5′, the tissue surrounding the prostate is heated intermittently while the prostate is heated continuously, as in all positions the bean stays focused on the prostate.

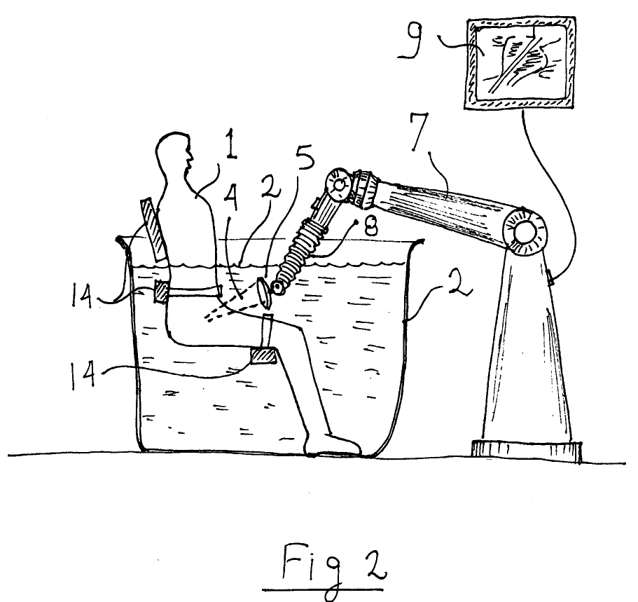

FIG. 2 shows the preferred embodiment of the invention. The transducer 5 is mounted on the arm of a robotic manipulator 7 that can be programmed to move transducer 5 around the target organ, such as prostate, in a manner always keeping the focal point on the target. The motion of robot arm 7 can be programmed to avoid directions passing through sensitive organs, such as the testes. The ultrasonic beam can be continuous or can be turned on for brief periods, for example the motion can stop momentarily while the beam is activated. Robot 7 and the software to run it are commercial products available from suppliers such as Fanuc or ABB. A sealing bellows 8 may be required for some robots. The patient 1 should be immobilized during the procedure by supports 14 or other means. A diagnostic system and monitor 9 is incorporated in the system. Ultrasonic coupling between the transducer and the patient other than a water bath can be used as well, such as local gels etc.

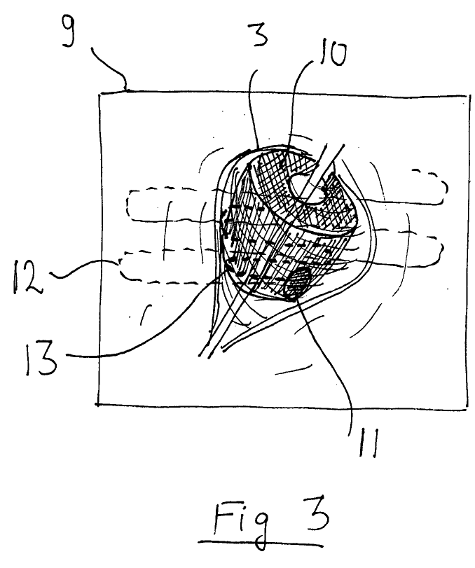

FIG. 3 schematically shows the operation of the diagnostic system in conjunction with the treatment system. The robot is programmed to first perform a 3D scan, at low power, of the target area. One possible scan pattern is shown as 12. The results of the scan are used to construct a 3D model of the target area such as prostate 3. The model is displayed on monitor 9. The doctor selects which part of the displayed area should be heated and marks the boundaries as a 3D model 10. The doctor also defines all the directions the beam should come from, eliminating mechanical obstacles and sensitive areas. The transducer is switched to high power and focal spot 11 performs a second 3D scan 13, within the pre-defined limits of model 10. The art of constructing 3D models from ultrasound data is well known and need not be detailed here. The software for covering a solid object with a scanned pattern and programming the robot to do so is readily available from the robot suppliers, as it is used in many industrial applications such as painting, polishing, grinding and machining. One review or such available software is given at: www.linuxdevices.com/articles/AT5739475111.html

Since the robot can be programmed for any scanning pattern the same system can be used to treat different cancers and medical conditions such as breast, liver, colon, bone and other cancers as well as heating non-malignant tissue. An example of treating non-malignant tissue is destroying or liquefying fat cells for cosmetic reasons.

1. A system for treating tissue with high intensity focused ultrasound by exposing said tissue to a focused ultrasound beam arriving from different angular directions at different times, all said direction passing through said tissue.

2. A high intensity focused ultrasound ablation system comprising a transducer mounted on a robotic manipulator, said manipulator capable of delivering an ultrasound beam from different angular directions having a common point of intersection at different times.

3. A system for heating undesired tissue by a beam of high intensity focused ultrasound while minimizing heating of adjacent tissue by continuously changing the direction of said beam.

4. A system as in claim 1 wherein said system also includes the ability to scan the said tissue with a lower energy beam.

5. A system as in claim 2 wherein said system also includes the ability to scan the area to be ablated by operating said transducer at a lower intensity.

6. A system as in claim 2 wherein said system also includes the ability to scan the area to be ablated using a second ultrasound transducer.

7. A system as in claim 1 wherein system is at least partially submerged in water while operating.

8. A system as in claim 2 wherein system is at least partially submerged in water while operating.

9. A system as in claim 3 wherein system is at least partially submerged in water while operating.

10. A system as in claim 1 wherein said tissue is a prostate.

11. A system as in claim 2 wherein system is used to treat the prostate of a patient placed inside a water tank.

12. A system as in claim 1 wherein said system is used to perform an ultrasound scan of said tissue in order to determine the boundaries of said exposure to high intensity ultrasound.

13. A system as in claim 2 wherein said manipulator is a robot.

14. A system as in claim 1 wherein direction passing through sensitive organs can be eliminated.

15. A system as in claim 3 wherein said tissue is a prostate.

16. A system as in claim 2 wherein motion of said system is stopped momentarily while beam is activated.

17. A system as in claim 1 wherein said tissue is a tumor.

18. A system as in claim 1 wherein said tissue is fat.

19. A system as in claim 1 used to treat breast cancer.

20. A system as in claim 1 wherein said tissue is a lung.
